(19) United States
(12) Patent Application Publication
(10) Pub. No.: US 2023/0355159 A1
(43) Pub. Date: Nov. 9, 2023

(54) DETECTING POTENTIAL SLOW-CONDUCTION CARDIAC TISSUE AREAS IN STABLE ARRHYTHMIAS

(71) Applicant: BIOSENSE WEBSTER (ISRAEL) LTD., Yokneam (IL)

(72) Inventors: Meytal Segev, Haifa (IL); Fady Massarwa, Baka Al Gharbiyya (IL); Sigal Altman, Ramat Hashofet (IL)

(21) Appl. No.: 17/736,292

(22) Filed: May 4, 2022

(51) Int. Cl. A61B 5/367; A61B 5/308; A61B 5/363; A61B 5/339

(52) U.S. Cl. CPC A61B 5/367; A61B 5/308; A61B 5/363; A61B 5/339

(57) ABSTRACT

A method for identifying candidate locations for ablation includes receiving an electrophysiological (EP) map comprising anatomical surface of cardiac chamber overlaid with (i) activation wave velocity vectors, (ii) data points comprising positions on surface and respective local activation times (LAT), and (iii) areas designated by early meet late (EML) LAT range. Set of shortest paths on cardiac surface is identified between different EML areas. One or more ranges of LAT values are selected, being characterized by lowest prevalence over data points of EP map. Complex tags are generated for positions having the LAT values within the one or more ranges of LAT values having lowest prevalence. Subset of the shortest paths is selected based on (i) density of complex tags along shortest paths and (ii) directions of activation wave velocity vectors relative to each of shortest paths. Selected subset of shortest paths are presented as candidate slow-conduction areas for ablation.

### FIELD OF THE DISCLOSURE

This disclosure relates generally to analysis of electrophysiological (EP) signals, and specifically to a method for detecting slow conduction in stable arrhythmias, such as an atrial flutter.

### BACKGROUND OF THE DISCLOSURE

Electrophysiological (EP) mapping of cardiac regions to identify cardiac tissue regions of slow conductions was previously proposed in the patent literature. For example, International Patent Application Publication WO 2020/214439 describes an electroanatomical mapping system that can map electrical activation of tissue, and in particular create a slow-conduction map, using a plurality of electrophysiology data points, each including local activation timing information, by computing a slow-conduction metric for each point using the local activation timing information. The slow-conduction metric can be used to classify points as no conduction points, slow-conduction points, and normal conduction points, and the results can be graphically expressed, including as an animated representation of an activation wavefront propagating along a three-dimensional anatomical surface model.

### BRIEF DESCRIPTION OF THE DRAWINGS

The present disclosure will be more fully understood from the following detailed description of the examples thereof, taken together with the drawings, in which:

FIG. 1 is a schematic, pictorial illustration of a catheter-based electrophysiological (EP) mapping system, according to an example of the present disclosure;

FIGS. 2A-2C are a schematic, pictorial illustrations of algorithm steps for the detection of slow-conduction cardiac tissue areas in stable arrhythmias, according to an example of the present disclosure;

FIG. 3 is a flow chart that schematically illustrates a method and algorithm for the detection of slow-conduction cardiac tissue areas in stable arrhythmias, according to an example of the present disclosure; and

FIG. 4 is a volume rendering of an EP map of a left atrium showing tissue locations of potential slow conduction that may cause a stable arrhythmia, according to an example of the present disclosure.

### DETAILED DESCRIPTION OF EXAMPLES

Overview A stable arrhythmia, such as an atrial flutter (AFL) or a stable ventricular tachycardia (VT), is often defined as a sustained arrhythmia not associated with significant hemodynamic compromise. However, a stable arrythmia can readily deteriorate into life-threatening conditions, and therefore, once diagnosed, it requires prompt treatment, including an option to ablate arrhythmogenic tissue locations that cause the stable arrhythmia.

In order to characterize a stable arrhythmia of a patient, a catheter-based electrophysiological (EP) mapping system may be used to generate an EP map of least part of the heart of the patient, such as an EP map of a cardiac chamber. In a typical catheter-based EP mapping procedure, a distal end of a catheter, which comprises one or more sensing electrodes, is inserted into the cardiac chamber to sense EP signals, such as bipolar electrograms (EGM). As a physician operating the system moves the distal end inside the cardiac chamber, the EP-mapping system acquires EP signals at various locations on the inner surface of the cardiac chamber, as well as the respective positions of the distal end. Based on these acquired signals, a processor of the mapping system generates the required EP map comprising local EP tissue characteristics (e.g., local cycle time between activations) overlaid on an anatomical map of the cardiac chamber.

When mapping a stable arrhythmia (e.g., an AFL), a physician looks for particular areas on a surface of cardiac tissue having aberrant EP characteristics, e.g., one or more tissue isthmuses, which are EP-conducting zones between adjacent scars or between natural structural barriers and scars. Such zones may be characterized by EP signals having slow conduction that can facilitate stable arrhythmias. The physician may therefore look on the EP map for slow-conduction zones and ablate one or more of these target zones to terminate an arrythmia. This may be used mostly in AFL but also potentially in VT cases.

However, slow-conduction areas are characterized by complex phenomenology, such as scar/line of blocked areas, synchronized wave direction, instability of local activation time (LAT), and low-amplitude bipolar signals, among others, which makes identification of most relevant slow-conduction areas difficult.

Present systems using current algorithms for identifying and characterizing a stable arrhythmia do not provide good pointers to the most likely areas for a physician to ablate, so the physician typically has to perform time-consuming checking of candidate tissue locations before ablation. In particular, current workflows to detect slow-conduction areas in AFL give no clear guidance for the most relevant features or workflow that should be used to enhance detection of those areas. Today, the physician needs to invest time to activate, configure and analyze a number of system features, and, in addition, manually tag a significant number of, for example, complex fractionated signals, as well as mark potential ablation sites.

Examples of the present disclosure that are described herein provide a method and system to provide an integrated automatic analysis that enables the user to easily detect potential slow-conduction areas. The indicated slow-conduction areas, detected by the algorithm using slow-conduction criteria comprising three steps described below, are marked (e.g., overlaid) on an EP map.

The disclosed algorithm relies on the observation that AFL and some other stable arrhythmias are caused by the occurrence of aberrant activation wave reentries due to the EP activation wave traveling in closed trajectories within the heart, rather than moving from one end of the heart to the other and then terminating. As the EP activation wave continuously propagates at some cycle length (e.g., a time between consecutive peaks in the ECG), wave propagation in a cardiac chamber can span the entire reentry cycle, where a “late” wavefront in the cycle meets the “early” wavefront of the next cycle. As a result, regions might be assigned incorrect local activation times (LATs) that are either very short or very long (a phenomenon called hereinafter “Early Meet Late,” or EML).

Typically, EML areas are bound by a curve of blocked conduction, such as curves described in U.S. Pat. No. 9,649,046, which is assigned to the assignee of the present application. EP map visualization is available to highlight such potential areas of blocked conduction, the potential areas of conduction block called hereinafter “EML areas,” and the encircling curves of blocked conduction called “EML boundaries.” An example of such a visualization of areas of blocked conduction (e.g., EML areas) in complex arrhythmias is disclosed in U.S. Pat. No. 10,136,828, which is assigned to the assignee of the present application.

In some examples of the disclosed technique, a processor receives an EP map of a cardiac surface, the EP map comprising data points comprising positions on the surface and respective local activation times (LAT), as well as areas designated by early meets late (EML) LAT occurrences. The processor identifies a set of shortest paths between different EML areas where such paths may cross isthmus zones. The processor does not consider exceedingly long paths, i.e., paths between remotely located EML areas, since they are unlikely to represent valid paths across isthmuses.

Using an LAT histogram of the surface, the processor selects one or more ranges (e.g., bins of a histogram) of LAT values with the lowest prevalence of such data points. The processor generates complex fractionated tags for the positions on the EP map having LAT values within these one or more ranges of LAT values with lowest prevalence. An algorithm that places complex tags at tissue locations according to magnitude of fractionations (indicative of stable arrhythmia) is described in U.S. patent application Ser. No. 17/548,558, titled, “Detection of Fractionated Signals in stable arrhythmias,” filed Dec. 12, 2021. The processor removes short paths along areas with sparse complex tags within them from consideration by selecting a subset of the set's shortest paths between the EML areas, based on density of complex fractionated tags along paths.

The EP map further includes activation wave velocities. The processor further filters the subset of the shortest paths between the EML areas by selecting paths whose velocity vectors in the isthmus areas propagating in a general direction along the isthmus, and not propagating between EML areas. The propagation along an isthmus is likely to be a source of reentrant arrhythmia, whereas propagation across an isthmus is likely a result of collision of EP wavefronts therein, which is clinically irrelevant.

Finally, the processor presents the selected subset of paths as candidate slow EP conduction gaps for ablation aimed at eliminating an arrhythmia (such as AFL).

System Description

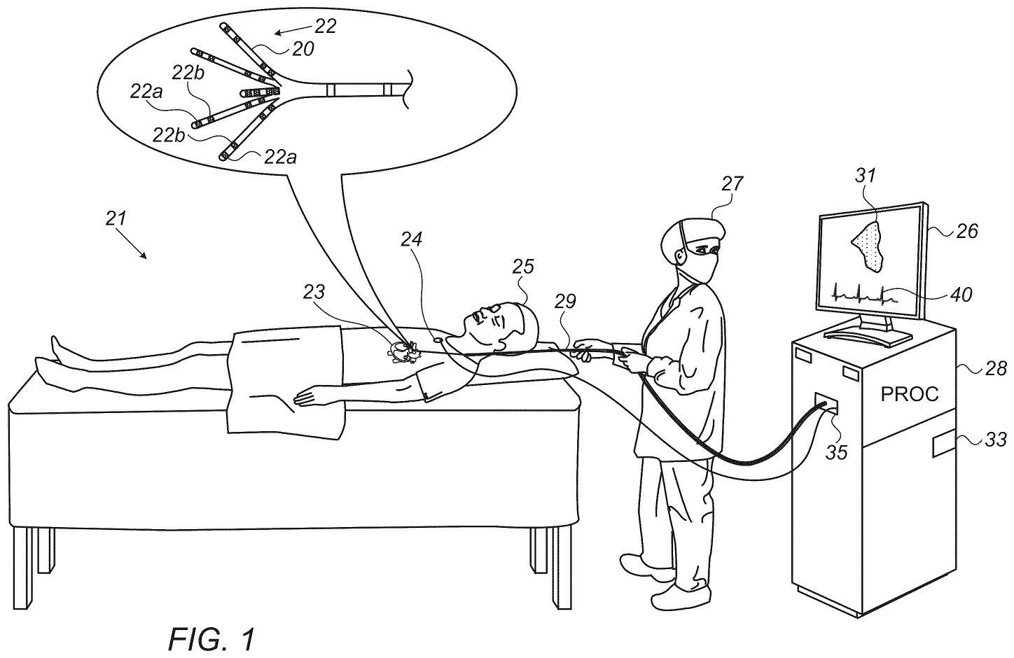

FIG. 1 is a schematic, pictorial illustration of a catheter-based electrophysiological (EP) mapping system 21, according to an example of the present disclosure. FIG. 1 depicts a physician 27 using an electro-anatomical mapping catheter 29 to perform an electro-anatomical mapping of a heart 23 of a patient 25. Mapping catheter 29 comprises, at its distal end, one or more arms 20, each of which is coupled to a bipolar electrode 22 comprising adjacent electrodes 22a and 22b. In some examples, the distal end of the catheter includes a magnetic sensor (not shown) that allows to magnetically track the locations of electrodes 22 or to calibrate the aforementioned the electrical tracking signals to improve an accuracy of an electrical location tracking method.

During the mapping procedure, the locations of electrodes 22 are tracked while they are inside heart 23 of the patient. For that purpose, electrical signals are passed between electrodes 22 and external electrodes 24. For example, three external electrodes 24 may be coupled to the patient's chest, and another three external electrodes may be coupled to the patient's back. For ease of illustration, only one external electrode is shown in FIG. 1.

Based on the signals, and given the known positions of electrodes 24 on the patient's body, processor 28 calculates an estimated location of each electrode 22 within the patient's heart. Respective electrophysiological data, such as bipolar electrogram traces, are additionally acquired from heart 23 tissue by using electrodes 22. The processor may thus associate any given signal received from electrodes 22, such as a bipolar EP signal, with the location at which the signal was acquired. The processor 28 receives the resulting signals via an electrical interface 35, and uses information contained in these signals, and stored in a memory 33, to construct an electrophysiological map 31 (that will also be stored in memory 33 for upload by processor 28) and EGM or ECG traces 40, and to present these on a display 26. One tracking system and method capable of producing map 31 is the Advanced Current Location (ACL) system, implemented in various medical applications, for example, in the CARTO™ system, produced by Biosense-Webster Inc., which is described in detail in U.S. Pat. No. 8,456,182 whose disclosure is incorporated herein by reference.

A magnetic tracking system (not shown) and method capable of producing map 31 or improving the accuracy of the ACL method is described in U.S. Pat. Nos. 5,391,199, 6,690,963, 6,484,118, 6,239,724, 6,618,612 and 6,332,089, in PCT Patent Publication WO 96/05768, and in U.S. Patent Application Publication Nos. 2002/0065455 A1, 2003/0120150 A1 and 2004/0068178 A1.

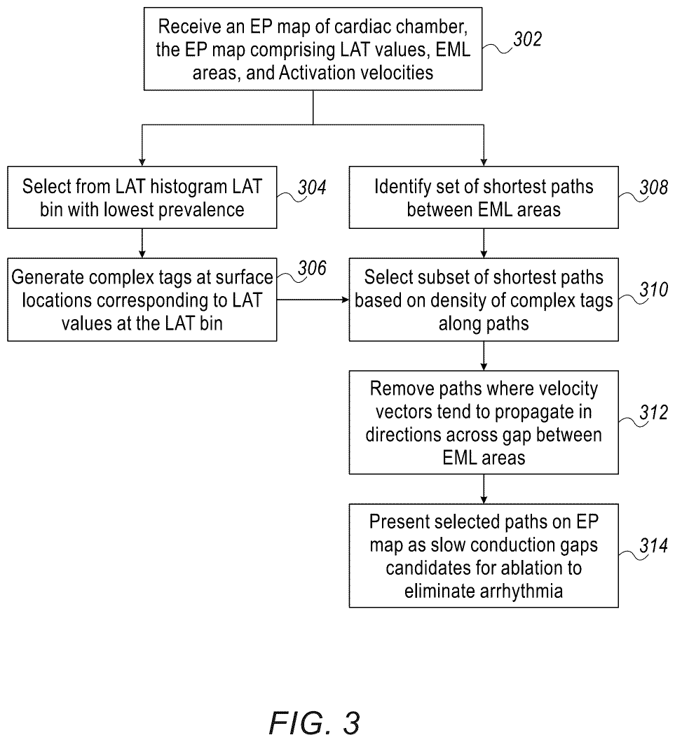

Processor 28 typically comprises a general-purpose computer with software programmed to carry out the functions described herein. The software may be downloaded to the computer in electronic form, over a network, for example, or it may, alternatively or additionally, be provided and/or stored on non-transitory tangible media, such as magnetic, optical, or electronic memory. In particular, processor 28 runs a dedicated algorithm as disclosed herein, including in FIG. 3, that enables processor 28 to perform the disclosed steps, as further described below.

The example illustration shown in FIG. 1 is chosen purely for the sake of conceptual clarity. Other types of electrophysiological sensing catheter geometries, such as the Lasso® Catheter (produced by Biosense-Webster Inc., Irvine, California) may be employed. Additionally, contact sensors may be fitted at the distal end of mapping catheter 29 to transmit data indicative of the physical quality of electrode contact with tissue. In an example, measurements of one or more electrodes 22 may be discarded if their physical contact quality is indicated as poor, and the measurements of other electrodes may be regarded as valid if their contact quality is indicated as sufficient.

Detection of Potential Slow-Conduction Cardiac Tissue Areas in Stable Arrhythmias

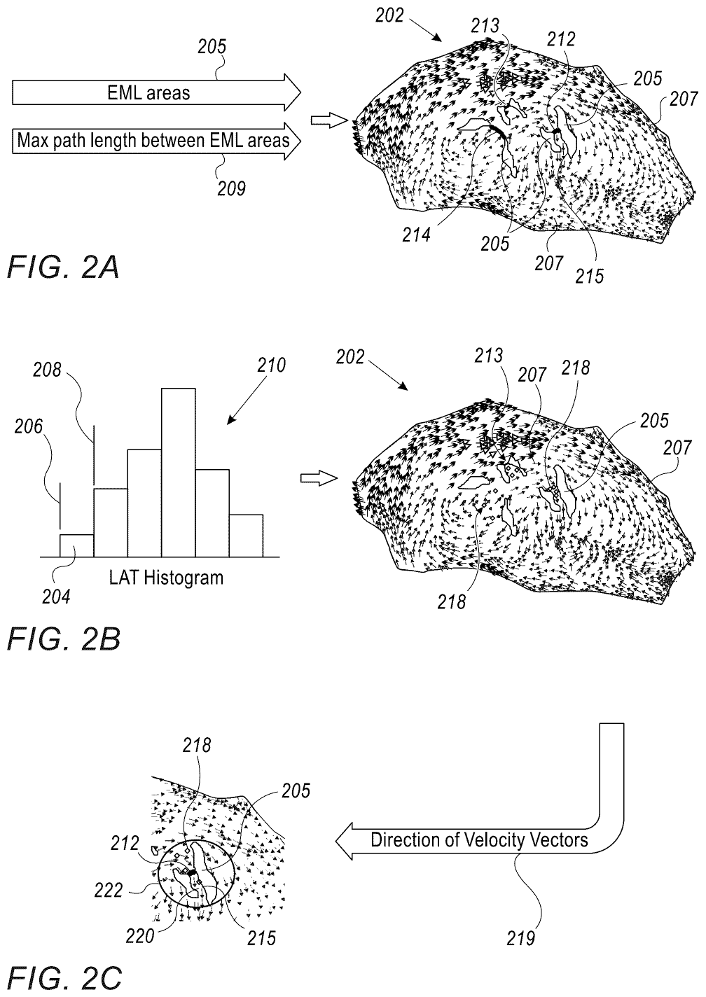

FIGS. 2A-2C are a schematic, pictorial illustrations of algorithm steps for the detection of slow-conduction cardiac tissue areas in stable arrhythmias, according to an example of the present disclosure.

FIGS. 2A-2C are a high-level visual description of the algorithm steps briefly described in the text above, and which are described in detail in FIG. 3.

FIG. 2A shows a set of shortest paths (212, 213, 214) identified on an EP map 202 by a processor using the disclosed algorithm. The paths are drawn over candidate slow-conduction gaps between EML areas 205. The inputs of this step comprise the EML areas 205, each defined by a curve around an area of blocked conduction, called herein an EML boundary 215, and by a predefined limit 209 on maximal path length considered (also called below “MAX_GAP_LENGTH”) to remove irrelevant (e.g., too long) paths, i.e., paths between remotely located EML areas, as described above and detailed in FIG. 3.

FIG. 2B shows that paths 214 are removed from consideration using LAT histogram 210, of which a lowest prevalence bin 204 is only considered (LAT bin 204 includes data ponits in EP map 202 within LAT range [minLAT 206, maxLAT 208]). As seen on EP map 202, only complex tags 218 that are associated with LAT bin 204, are considered (i.e., filtered), which leaves for consideration only paths based on density of the relevant complex tags 218 along each path of the group of paths (212, 213).

Finally, FIG. 2C shows a selected path 212, which is left on EP map 202 and indicated (222) to a user as a path over possible slow-conduction gap 220. Path 212 is left after filtering out paths, such as filtering out (219) path 213, aside which activation wave velocities 207 show propagation largely across the gap, rather than being more perpendicular or with an angle relative to path 212, since only the latter is indicative of possible reentrant activation via a gap (e.g., isthmus) 220 between close EML areas 205. In other words, FIG. 2C shows path selection based on filtering out paths for which the velocity vectors are parallel to a tangent at the path, up to a given angular tolerance over the degree of parallelism. On the other hand, velocity vectors that are largely orthogonal to the path (i.e., parallel to a tangent of the EML boundary) at the EML boundary 215, where the path meets the EML area, indicate reentrant propagation that may cause an arrhythmia.

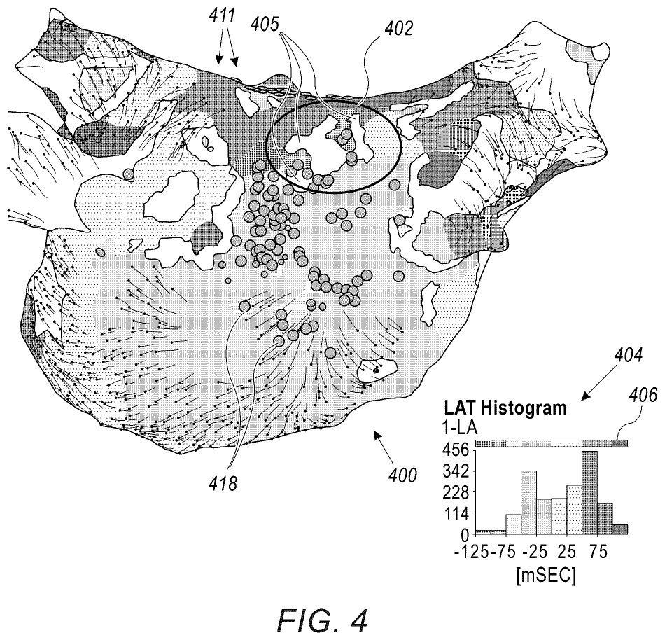

FIG. 3 is a flow chart that schematically illustrates a method and algorithm for the detection of slow-conduction cardiac tissue areas 220 in stable arrhythmias, according to an example of the present disclosure. The algorithm, according to the presented example, carries out a process that begins at an EP map receiving step 302, with a processor, such as processor 28, receiving an EP map, such as map 202, comprising (i) surface locations and respective LAT values at the locations, (ii) activation wave velocity vectors (e.g., vectors 207), and EML areas (e.g., areas 205). The surface location and LAT values are not shown explicitly in the map, rather, as shown by the EP map of FIG. 4, such EP maps are typically color coded with a continuous LAT scale interpolating between the discrete LAT values measured.

Next, the process runs steps 304 and 306 in parallel to step 308, to generate both necessary inputs for step 310.

At an LAT bin selection step 304, the processor selects one or more LAT bins of a LAT histogram of the LAT values of step 302. The processor may select a lowest prevalence LAT bin, such as bin 204 of FIG. 2B, or, for example, select both lowest and second-lowest prevalence LAT bins. Step 304 may involve calculating the LAT histogram, if such is not already available.

Next, at a complex tags generation step 306, processor 28 applies the aforementioned complex tags algorithm of U.S. patent application Ser. No. 17/548,558 with the [minLAT, maxLAT] bin 204 as the “time frame within WOI” to identify locations of such a subset of tags on the map surface.

In parallel, at paths identification step 308, processor 28 finds virtual shortest paths (212, 213, 214) between all EML areas 205. As a preparatory step, processor 28 utilizes an existing algorithm to find all EML areas 205, if such are not already given on EP map 202. Step 308 further excludes paths with lengths exceeding a predefined maximal path length, “MAX_GAP_LENGTH,” to remove irrelevant (e.g., too long) paths which are therefore not paths over isthmuses.

At a path subset selection step 310, the processor further filters out irrelevant paths, based on a low density of tags along a path (e.g., within an area including the path).

At a final path selection step 312, processor 28 maintains only paths that meet a last step in the slow-conduction criteria, by maintaining paths for which velocity vectors along the EML boundary are consistent in direction and are parallel to the EML boundary tangent direction up to a predefined parallelism tolerance.

Finally, at paths presentation step 314, processor 28 presents the selected subset of paths as candidate slow-conduction gaps for ablation aimed at eliminating the cardiac arrhythmia.

The flow chart shown in FIG. 3 is chosen purely for the sake of conceptual clarity. Other possible steps are omitted from the disclosure herein purposely in order to provide a more simplified flow chart.

FIG. 4 is a volume rendering 400 of an EP map of a left atrium showing tissue locations of potential slow conduction that may cause a stable arrhythmia, according to an example of the present disclosure. As seen, the processor indicates (402) a region of isthmuses between EML areas 405, that are identified as crossed by paths satisfying the above described three elements of slow-conduction criteria:
1. High density of complex tags 418 in path area
2. Path length<MAX_GAP_LENGTH
3. Velocity vectors along the paths consistent in direction and not parallel to the paths' tangent direction

As seen, other possible isthmuses causing arrhythmia, such as isthmuses at regions 411, are automatically ruled out by the disclosed technique.

FIG. 4 further shows an LAT histogram 404, which is used in filtering designed to maintain only relevant complex tags 418, as described above. Further shown is a continuous LAT scale 406 of EP map 400 and histogram 404, which the physician may use to verify locations of late activation times which are indicative of an arrythmia, such as those found inside the region emphasized (402) by the disclosed technique, to consider ablation therein.

### EXAMPLES

### Example 1

A method for identifying candidate locations for ablation includes receiving an electrophysiological (EP) map (202) comprising an anatomical surface of a cardiac chamber overlaid with (i) activation wave velocity vectors (207), (ii) data points comprising positions on the surface and respective local activation times (LAT), and (iii) areas (205) designated by early meet late (EML) LAT range. A set of shortest paths (212, 213, 214) on the cardiac surface is identified between different EML areas (205). One or more ranges (204) of LAT values are selected, that are characterized by lowest prevalence over the data points of the EP map. Complex tags (218) are generated for the positions having the LAT values within the one or more ranges (204) of LAT values having the lowest prevalence. A subset of the shortest paths (212) is selected based on (i) density of the complex tags (218) along the shortest paths and (ii) directions of the activation wave velocity vectors (207) relative to each of the shortest paths. The selected subset of the shortest paths (212) is presented as candidate slow-conduction areas for ablation.

### Example 2

The method according to example 1, wherein selecting the one or more ranges of the LAT values comprises selecting one or more LAT bins (204) of a LAT histogram (210).

### Example 3

The method according to example 1, wherein selecting the subset of the shortest paths (212, 213) comprises filtering out a path (214) longer than a predefined path length.

### Example 4

The method according to any of examples 1 through 3, wherein receiving the EP map (202) with the areas (205) designated by early meet late (EML) LAT range comprises receiving areas (205) bounded by a curve (215) of blocked conduction.

### Example 5

The method according to any of examples 1 through 4, wherein selecting the shortest paths based on the direction of the activation wave velocity vectors (207) comprises filtering out a path (213) for which the activation wave velocity vectors are parallel to a tangent to the path (213), up to a given angular tolerance.

### Example 6

The method according to claim 1, wherein generating the complex tags (218) comprises annotating electrograms at fractioned regions, and extracting LAT values using the annotated electrograms.

### Example 7

The method according to claim 1, wherein the cardiac chamber is an atrium and the arrhythmia is an atrial flutter.

### Example 8

The method according to claim 1, wherein the cardiac chamber is a ventricle and the arrhythmia is ventricular tachycardia.

### Example 9

A system for identifying candidate locations for ablation, the system comprising an interface (35) and a processor (28). The interface (35) is configured receive an electrophysiological (EP) map comprising an anatomical surface of a cardiac chamber overlaid with (i) activation wave velocity vectors (207), (ii) data points comprising positions on the surface and respective local activation times (LAT), and (iii) areas (205) designated by early meet late (EML) LAT range. The processor (28) is configured to (a) identify a set of shortest paths (212, 213, 214) on the cardiac surface between different EML areas (205), (b) select one or more ranges (204) of LAT values that are characterized by lowest prevalence over the data points of the EP map, (c) generate complex tags (218) for the positions having the LAT values within the one or more ranges (204) of LAT values having the lowest prevalence, (d) select a subset of the shortest paths (212) based on (i) density of the complex tags (218) along the shortest paths and (ii) directions of the activation wave velocity vectors (207) relative to each of the shortest paths, and (e) present the selected subset of the shortest paths (212) as candidate slow-conduction areas for ablation.

Although the examples described herein mainly address cardiac diagnostic applications, the methods and systems described herein can also be used in other medical applications.

It will be appreciated that the examples described above are cited by way of example, and that the present disclosure is not limited to what has been particularly shown and described hereinabove. Rather, the scope of the present disclosure includes both combinations and subcombinations of the various features described hereinabove, as well as variations and modifications thereof which would occur to persons skilled in the art upon reading the foregoing description and which are not disclosed in the prior art.

1. A method for identifying candidate locations for ablation, the method comprising:
receiving an electrophysiological (EP) map comprising an anatomical surface of a cardiac chamber overlaid with (i) activation wave velocity vectors, (ii) data points comprising positions on the surface and respective local activation times (LAT), and (iii) areas designated by early meet late (EML) LAT range;
identifying a set of shortest paths on the cardiac surface between different EML areas;
selecting one or more ranges of LAT values that are characterized by lowest prevalence over the data points of the EP map;
generating complex tags for the positions having the LAT values within the one or more ranges of LAT values having the lowest prevalence;
selecting a subset of the shortest paths based on (i) density of the complex tags along the shortest paths and (ii) directions of the activation wave velocity vectors relative to each of the shortest paths; and
presenting the selected subset of the shortest paths as candidate slow-conduction areas for ablation.

2. The method according to claim 1, wherein selecting the one or more ranges of the LAT values comprises selecting one or more LAT bins of a LAT histogram.

3. The method according to claim 1, wherein selecting the subset of the shortest paths comprises filtering out a path longer than a predefined path length.

4. The method according to claim 1, wherein receiving the EP map with the areas designated by early meet late (EML) LAT range comprises receiving areas bounded by a curve of blocked conduction.

5. The method according to claim 1, wherein selecting the shortest paths based on the direction of the activation wave velocity vectors comprises filtering out a path for which the activation wave velocity vectors are parallel to a tangent to the path, up to a given angular tolerance.

6. The method according to claim 1, wherein generating the complex tags comprises annotating electrograms at fractioned regions, and extracting LAT values using the annotated electrograms.

7. The method according to claim 1, wherein the cardiac chamber is an atrium and the arrhythmia is an atrial flutter.

8. The method according to claim 1, wherein the cardiac chamber is a ventricle and the arrhythmia is ventricular tachycardia.

9. A system for identifying candidate locations for ablation, the system comprising:
an interface configured receive an electrophysiological (EP) map comprising an anatomical surface of a cardiac chamber overlaid with (i) activation wave velocity vectors, (ii) data points comprising positions on the surface and respective local activation times (LAT), and (iii) areas designated by early meet late (EML) LAT range; and
a processor, which is configured to:
identify a set of shortest paths on the cardiac surface between different EML areas;
select one or more ranges of LAT values that are characterized by lowest prevalence over the data points of the EP map;
generate complex tags for the positions having the LAT values within the one or more ranges of LAT values having the lowest prevalence;
select a subset of the shortest paths based on (i) density of the complex tags along the shortest paths and (ii) directions of the activation wave velocity vectors relative to each of the shortest paths; and
present the selected subset of the shortest paths as candidate slow-conduction areas for ablation.

10. The system according to claim 9, wherein the processor is configured to select the one or more ranges of the LAT values by selecting one or more LAT bins of a LAT histogram.

11. The system according to claim 9, wherein the processor is configured to select the subset of the shortest paths by filtering out a path longer than a predefined path length.

12. The system according to claim 9, wherein the interface is configured to receive the EP map with the areas designated by early meet late (EML) LAT range by receiving areas bounded by a curve of blocked conduction.

13. The system according to claim 9, wherein the processor is configured to select the shortest paths based on the direction of the activation wave velocity vectors by filtering out a path for which the activation wave velocity vectors are orthogonal to a tangent to the path, up to a given angular tolerance.

14. The system according to claim 9, wherein the processor is configured to generate the complex tags by annotating electrograms at fractioned regions, and extracting LAT values using the annotated electrograms.

15. The system according to claim 9, wherein the cardiac chamber is an atrium and the arrhythmia is an atrial flutter.

16. The system according to claim 9, wherein the cardiac chamber is a ventricle and the arrhythmia is ventricular tachycardia.
